(19) United States
(12) Patent Application Publication
(10) Pub. No.: US 2011/0190270 A1
(43) Pub. Date: Aug. 4, 2011

(54) METHODS OF TREATING THROMBOEMBOLIC DISORDERS

(75) Inventors: Shaun Jackson, Victoria (AU); Simone Schoenwaelder, Victoria (AU); Akiko Ono, Victoria (AU)

(73) Assignee: MONASH UNIVERSITY, Clayton, Victoria (AU)

(21) Appl. No.: 12/865,375

(22) PCT Filed: Jan. 30, 2009
(86) PCT No.: PCT/AU09/00105

§ 371 Date: Apr. 19, 2011

(30) Foreign Application Priority Data
Jan. 31, 2008 (AU) 2008900430
Feb. 1, 2008 (AU) 2008900464

(51) Int. Cl. A61K 31/551; A61K 31/44; A61K 31/437; A61P 7/02

(52) U.S. Cl. 514/218; 514/357; 514/300

(57) ABSTRACT

The field of the invention relates to methods for dissolving a thrombus using inhibitors of platelet contractility. More particularly, the present invention relates to the use of an inhibitor of platelet contractility in combination with one or more thrombolytic agents and optionally one or more anticoagulants for inhibiting platelet contraction and consolidation in the developing thrombus.

### FIELD OF THE INVENTION

The field of the invention relates to methods for dissolving a thrombus using inhibitors of platelet contractility. More particularly, the present invention relates to the use of an inhibitor of platelet contractility in combination with one or more thrombolytic agents and optionally one or more anticoagulants for inhibiting platelet contraction and consolidation in the developing thrombus.

### BACKGROUND OF THE INVENTION

Thrombus formation can be divided into two temporally district phases. The first phase being the formation of a primary hemostatic plug, composed of aggregated platelets, forming independently of fibrin generation. This primary platelet plug (or thrombus) is consolidated during the secondary hemostatic phase, when fibrin polymers then become enmeshed within the developing thrombus to physically stabilise the platelet plug. During the development of the hemostatic plug, platelets undergo a complex series of morphological and functional responses that require extensive remodelling of the actin cytoskeleton. These cytoskeletal changes are indispensable for the normal hemostatic function of platelets and are controlled by a complex network of signalling, structural and regulatory proteins.

The actin-based cytoskeleton of platelets can be separated into two functionally distinct structures; (i) the spectrin-rich membrane skeleton; lining the inner plasma membrane, and the (ii) cytoskeleton; consisting of long actin filaments that radiate from the cell centre to the surface membrane. The membrane skeleton is essential for maintaining the structure and integrity of the surface membrane, whereas the cytoskeleton, through its attachment to myosin, principally generates contractile forces within the cell. The internal generation of contractile force has a role in regulating platelet shape change¹ and in promoting granule secretion², whereas the external transmission of contractile force is essential for fibrin clot retraction³ which occurs during the secondary hemostatic phase.

Platelet contractility requires phosphorylation of myosin light chains (MLC), which is under the dual control of myosin light chain kinase (MLCK) and myosin phosphatase (mPP). In platelets, calcium appears to be the predominant regulator of contractile force generation, as inhibition of Rho kinase has been found to have a minimal effect on the

fibrin clot retraction phase⁴ and only inhibits platelet shape change under experimental conditions limiting cytosolic calcium flux.

During the second phase of thrombus formation, the platelet-fibrin complex undergoes retraction (referred to as “fibrin clot retraction” phase) which is designed to assist in stabilising the thrombus. Rho kinase plays a role in regulating the stability of platelet-platelet adhesion contacts during the initial development of a thrombus since inhibition of Rho kinase undermines the stability of platelet-matrix and platelet-platelet interactions in a shear field⁵, leading to a major defect in thrombus growth⁶.

Studies on mice with a targeted deletion of myosin IIA in platelets have confirmed the importance of the platelet contractile mechanism in supporting the hemostatic function of platelets, leading to a major prolongation in tail bleeding time and a severe defect in thrombus growth. Complete deficiency of myosin IIa abolished platelet shape change and clot retraction however platelet aggregation and granule release largely remains intact.

It is not clear however, how important contractility is to the regulation of the primary hemostatic plug, independent of blood coagulation and fibrin clot retraction.

### SUMMARY OF THE INVENTION

The present invention provides a method for dissolving a thrombus in a subject, comprising administering to the subject a platelet contractility inhibitor in combination with one or more thrombolytic agents and optionally one or more anticoagulants.

The present invention also provides a method for inhibiting thrombus contraction in a subject, comprising administering to the subject a platelet contractility inhibitor, in combination with one or more thrombolytic agents and optionally one or more anticoagulants.

The present invention also provides a method for enhancing the effectiveness of a thrombolytic agent, comprising administering to the subject a platelet contractility inhibitor together with the thrombolytic agent at a time when the thrombus is forming or has formed from aggregated platelets.

The present invention also provides for the use of a platelet contractility inhibitor, in combination with one or more thrombolytic agents and optionally one or more anticoagulants for inhibiting thrombus contraction in a subject.

In one embodiment of the invention, the platelet contractility inhibitor is administered locally at the site where the thrombus has formed.

In another embodiment of the invention, the platelet contractility inhibitor is administered directly into the thrombus.

In another embodiment of the invention, the platelet contractility inhibitor is administered as a bolus.

In another embodiment of the invention, the platelet contractility inhibitor is administered as an oral or intravenous bolus or as a bolus plus infusion to maintain inhibition at steady-state levels.

In one embodiment of the invention, the platelet contractility inhibitor is administered according to the invention to the subject within 12 hours after the first identification of a thromboembolic disorder.

In another embodiment of the invention, the platelet contractility inhibitor is administered according to the invention to the subject within 3 hours after the first identification of a thromboembolic disorder.

In another embodiment of the invention, the platelet contractility inhibitor is administered according to the invention to a subject within 3 hours of a stroke.

In another embodiment, the platelet contractility inhibitor is administered according to a method of the invention to the subject immediately after a stroke.

In another embodiment of the invention, the platelet contractility inhibitor is administered according to the invention to the subject within 3 hours of a heart attack.

In one embodiment of the invention, the platelet contractility inhibitor is a Rho kinase inhibitor. In another embodiment of the invention, the platelet contractility inhibitor is blebbistatin. In another embodiment of the invention, the platelet contractility inhibitor is a Rho inhibitor.

The Rho kinase inhibitor is preferably selected from the group consisting of:

(i) Isoquinolinesulfonamides such as (S)-(+)-2-methyl-1-[(4-methyl-5-isoquinolinyl)sulfonyl]homopiperazine (dimethylfasudil) or 1-(5-isoquinolinesulfonyl)homopiperazine (fasudil) or salts thereof;
(ii) (+)-(R)-trans-4-(1-aminoethyl)-N-(4-pyridyl)cyclohexanecarboxamide or salts thereof;
(iii) (+)-(R)-trans-4-(1-aminoethyl)-N-(1H-pyrrolo[2,3-b]pyridine-4-yl)cyclohexanecarboxamide] or salts thereof; or
(iv) derivatives having Rho kinase inhibitory activity.

In one embodiment the salt is a hydrochloride.

The Rho inhibitor according to the invention is preferably an inhibitor of Rho GTPases. Preferably, the Rho inhibitor is selected from the group consisting of inhibitors of Cdc42, Rac1 and RhoA. In one embodiment, the Rho inhibitor is C3 transferase.

The platelet contractility inhibitor may be administered sequentially or concurrently with the one or more thrombolytic agents and optionally one or more anticoagulants.

Examples of suitable thrombolytic agents according to the invention include streptokinase (kabikinase, STREPTASE®), anistreplase (EMINASE®), urokinase (abbokinase), tenecteplase (TNKase, METALYSE®), reteplase (RETAVASE®, RAPILYSIN®) or tissue plasminogen activator (t-PA, alteplase, ACTIVASE®, ACTILYSE®). However, it would be appreciated by a person skilled in the art of the present invention that other thrombolytic agents not mentioned above would also be suitable for use in the invention.

The invention also provides for a dose of thrombolytic agent when used in combination with the platelet contractility inhibitor and optionally anticoagulant that is at, or lower than the dose prescribed according to the approved indications.

For example, in one embodiment, the platelet contractility inhibitor is administered in combination with a thrombolytic and optionally one or more anticoagulants, wherein the total dose of thrombolytic is less than 90 mg in a human subject. Preferably, the total dose of thrombolytic is less than 70 mg, more preferably less than 50 mg, still more preferably less than 35 mg, still more preferably less than 20 mg, even more preferably less than 10 mg.

In another embodiment, the platelet contractility inhibitor is administered in combination with t-PA and optionally one or more anticoagulants, wherein the total dose of t-PA is less than 90 mg, preferably less than 70 mg, more preferably less than 50 mg, still more preferably less than 35 mg, still more preferably less than 20 mg, even more preferably less than 10 mg.

In another embodiment, the platelet contractility inhibitor is administered in combination with streptokinase and optionally one or more anticoagulants, wherein the dose of streptokinase is less than 1,500,000 IU.

In another embodiment, the platelet contractility inhibitor is administered in combination with urokinase and optionally one or more anticoagulants, wherein the total dose of urokinase is less than 500,000 IU.

The invention also provides a method of treating a thromboembolic disorder, comprising administering to a subject a platelet contractility inhibitor, in combination with one or more thrombolytic agents and optionally one or more anticoagulants.

Examples of thromboembolic disorders according to the invention include ischemic stroke, acute myocardial infarction, deep vein thrombosis (DVT), pulmonary embolus, clotted AV fistula and shunts. It should be appreciated however, that is not an exhaustive list of thromboembolic disorders that may be treated.

The invention also provides a method of treating stroke, comprising administering to a subject a platelet contractility inhibitor, in combination with one or more thrombolytic agents and optionally one or more anticoagulants.

The invention also provides a method of treating a heart attack, comprising administering to a subject a platelet contractility inhibitor, in combination with one or more thrombolytic agents and optionally one or more anticoagulants.

The invention also provides use of a platelet contractility inhibitor, in combination with one or more thrombolytic agents and optionally one or more anticoagulants in the manufacture of a medicament for treating a thromboembolic disorder.

The invention also provides use of a platelet contractility inhibitor, in combination with one or more thrombolytic agents and optionally one or more anticoagulants in the manufacture of a medicament for treating stroke.

The invention also provides use of a platelet contractility inhibitor, in combination with one or more thrombolytic agents and optionally one or more anticoagulants in the manufacture of a medicament for treating heart attack.

The invention also provides a composition for use in dissolving a thrombus, the composition comprising a platelet contractility inhibitor and one or more thrombolytic agents.

The invention also provides a composition for use in dissolving a thrombus, the composition comprising a platelet contractility inhibitor, and one or more thrombolytic agents and one or more anticoagulants.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE FIGURES

FIG. 1. Fibrin-Independent Thrombus Contraction.

Human whole blood was collected in the absence of anticoagulant (Native) (A), in the presence of the fibrin polymerisation inhibitor GPRP (GPRP—280 μM) (A), or in the presence of Lepirudin (800 U/ml) (A,B), then perfused through Type I collagen-coated glass microslides at 1800 s′ for up to 5 minutes. (A) To visualize fibrin formation, whole blood perfusion was performed in the presence of Oregon-green labelled fibrinogen (20 μg/ml). DIC and fluorescence images were captured in real time using a Leica inverted microscope (63× mag). Images are taken from one representative of four independent experiments. (B) Thrombus formation and consolidation during perfusion of lepirudin-anticoagulated whole blood was recorded in real-time using DIC microscopy, and snap-shots of individual thrombi at the indicated time points were obtained off-line. These images are taken from 1 representative of 12 independent experiments. The original outline of the thrombus prior to contraction is highlighted by the broken line.

FIG. 2. Characterisation of Thrombus Consolidation In Vitro.

Lepirudin-anticoagulated human whole blood was perfused through collagen-coated microslides at 1,800 s⁻¹. A. To quantify thrombus volume, whole blood was preincubated with DiIC₁₂ prior to perfusion, and 3-D images captured in real-time using an inverted Leica DMIRB confocal microscope, followed by off-line analysis to quantify thrombus volume, as described in “three dimensional volumetric thrombus analysis”. This graph depicts thrombus volume over time from 4 individual thrombi taken from 4 independent experiments. B. Quantification of thrombus consolidation was performed by ‘spiking’ whole blood with DiIC₁₂-labelled platelets prior to perfusion, followed by capture of consecutive DIC and fluorescence images in real-time. (i) Images are taken from one flow representative of 12 independent flows. (ii) Off-line analysis was performed as described under “Two dimensional quantification of thrombus consolidation”, and results are expressed as percentage decrease in the distance between 2 fluorescently-labelled platelets incorporated into a thrombus prior to 1 minute of flow, with the distance between platelets at 1 minute taken as 100%. Results are expressed as the mean±SD of 36 individual thrombi, from 12 independent experiments (n=12) (solid line).

FIG. 3. Role of Calcium in Regulating Thrombus Contraction.

Lepirudin-anticoagulated human whole blood, spiked with DiIC₁₂-labelled platelets, was perfused through collagen-coated microslides at 1,800 s⁻¹. The decrease in distance between firmly adherent platelets was quantified as described in “Two-dimensional quantification of thrombus consolidation” and used as an indirect marker of thrombus contraction. (A) Whole blood was perfused in the presence of EGTA/Mg²⁺ (2 mM/1 mM). (B) For studies with 2-APB, whole blood was initially perfused for 30 sec without inhibitor to allow for the initial formation of non-contracted thrombi (refer to “Two-dimensional quantification of thrombus consolidation”), followed by perfusion of whole blood in the presence of 2-APB (200 μM). (A,B) Results represent the mean±SEM (n=5) (* p<0.05; ** p<0.005; *** p<0.001) and represent the % decrease in inter-platelet distance relative to 100% at 1 min. (C) To examine the importance of calcium flux for fibrin clot retraction, PRP was preincubated with 2-APB (200 μM), c7E3 (50 μg/ml) or EGTA/Mg²⁺ (2 mM/1 mM), followed by addition of thrombin (1 U/ml). Clot retraction was assessed as described under “Platelet-mediated fibrin clot retraction”. Results represent the mean±SEM (n=3) (ns=p>0.05; ** p<0.005).

FIG. 4. Role of Rho Kinase in Regulating Thrombus Contraction.

(A) Lepirudin-anticoagulated human whole blood, spiked with DiIC₁₂-labelled platelets, was preincubated with vehicle (DMSO), H1152 (40 μM) or HA1077 (80 μM), prior to perfusion through collagen-coated microslides at 1,800 s⁻¹. The distance between firmly adherent platelets was quantified and utilized as an indirect marker of thrombus contraction. Results represent the mean±SEM (n=4) (* p<0.05; ** p<0.005; *** p<0.001) and represent the % decrease in inter-platelet distance relative to 100% at 1 min. (B) Thrombus formation in the presence of vehicle (DMSO) or H1152 (40 μM) was recorded in real time, and snap shots of individual thrombi at the indicated times were taken off-line. The original size of the thrombus is outlined by a solid line, while the resultant thrombus size following 2 minutes of flow is outlined by a broken line. These images are taken from one representative of 4 independent experiments. (C) To examine the effect of Rho kinase inhibitors on fibrin-dependent clot retraction, citrated PRP was preincubated with vehicle (DMSO), H1152 (40 μM), HA1077 (80 μM) or c7E3 (100 μg/ml), followed by addition of thrombin (1.0 U/ml). The extent of clot retraction was assessed after 30 minutes, as described under “Platelet-mediated fibrin clot retraction”. Results represent the mean±SEM (n=3).

FIG. 5. Effect of the Myosin II Inhibitor (Blebbistatin) on Thrombus Consolidation In Vitro.

Lepirudin-anticoagulated human whole blood was preincubated with Blebbistatin (Blebbistatin [−]), or an inactive enantiomer of Blebbistatin (Blebbistatin [+]), prior to perfusion through collagen-coated microslides at 1,800 s⁻¹, as described in “Two dimensional quantification of thrombus consolidation”. (A) Snap-shots of individual thrombi over a time period of 1.5 minutes were obtained off-line. These images are taken from one representative of 3 independent experiments. (B) Whole blood spiked with DiIC₁₂-labelled platelets was perfused through collagen-coated microslides at 1,800 s⁻¹, and the distance between firmly adherent platelets quantified, as described in “Two-dimensional quantification of thrombus consolidation”. Results represent the mean±SEM (n=3) (ns p>0.05; ** p<0.005; *** p<0.001) and represent the % decrease in inter-platelet distance relative to 100% at 1 min.

FIG. 6. Role of Myosin IIa and Rho Kinase in Regulating Thrombus Stability in vivo.

Vascular injury was induced in the mesenteric post-capillary venules of anaesthetised C57/B16 mice by needle puncture, and thrombus development recorded as described in “intravital microscopy”. In the indicated experiments, the effects of an inactive entaniomer of blebbistatin (Blebbistatin [+]), vehicle (DMSO), H1152 or Blebbistatin (Blebbistatin [−]) on thrombus stability was assessed following intermittent injections (denoted by solid bars), with concentrations and volumes as described in “intravital microscopy”. (A) The relative change in surface area of a given thrombus over time was determined offline, and expressed relative to the initial surface area of the thrombus prior to injection. These results depict data taken from 1 of 4 independent experiments, with representative images from one such experiment depicted in (B). The percentage decrease in thrombus surface area following injection was quantified as described in “intravital microscopy”, and expressed as a percentage (%) of the original thrombus. These results represent the mean±SEM (n=4), where *** p<0.0001.

FIG. 7. Role of Myosin IIa and Rho Kinase in Regulating the Stability of the Primary Hemostatic Plug.

Vascular injury was induced in the mesenteric post-capillary venules of anaesthetised C57/B16 mice by needle puncture, in the presence of lepirudin (50 mg/kg, i.v.—administered prior to injury). In the indicated experiments, the effects of an inactive entaniomer of blebbistatin (Blebbistatin [+]), vehicle (DMSO), H1152 or Blebbistatin (Blebbistatin [−]) on thrombus stability was assessed following repetitive injections (denoted by solid bars), with concentrations and volumes as described in “intravital microscopy”. (A) The relative change in surface area of a given thrombus over time was determined offline, and expressed relative to the initial surface area of the thrombus prior to injection. These results depict data taken from 1 of 4 independent experiments, with representative images from one such experiment depicted in (B). (C) The maximum percentage decrease in thrombus size following each infusion of vehicle/inhibitor was quantified as described in “intravital microscopy”, and expressed as a percentage (%) of the original thrombus prior to infusion. These results represent the mean±SEM (n=4), where *** p<0.0001.

FIG. 8. Effect of Rho Kinase Inhibitors in Combination with t-PA or Urokinase±Anticoagulants on Vascular Reperfusion

Bar graphs (i) through (vi) demonstrate the effects of Rho kinase inhibitors (HA1077 and Y27632) in combination with t-PA or urokinase with or without anticoagulants on vascular perfusion in the carotid artery of anaesthetised mice. The various treatment regimens were as follows: A: saline, B: HA1077 (8 mg/kg), C: t-PA (2 mg/kg) bolus+18 mg/kg/30 min infusion, D: t-PA (2 mg/kg) & heparin (71 U/kg) boluses+t-PA (18 mg/kg/30 min) & heparin 28.6 U/kg/30 min infusion, E: t-PA (2 mg/kg) & heparin (142 U/kg) boluses+t-PA (18 mg/kg/30 min) heparin (57.2 U/kg/30 min) infusion, F: Y27632 (8 mg/kg) & t-PA (2 mg/kg) & heparin (142 U/kg) boluses+t-PA (18 mg/kg/30 min) & heparin (57.2 U/kg/30 min) infusion, G: HA-1077 (8 mg/kg) & urokinase (4,400 IU/kg) & heparin (142 U/kg) boluses+urokinase (4,400 IU/kg/30 min) & heparin (57.2 U/kg/30 min) infusion, H: HA1077 (8 mg/kg) & t-PA (2 mg/kg) & hirudin (10 mg/kg) boluses+t-PA 18 mg/kg/30 min) infusion. Solid black bars=No reperfusion, Striped bars=Unstable reperfusion—refers to an intermittent flow disturbance, characterised by periods of normal flow interspersed with periods of re-occlusion, Sold grey bars=Moderately stable reperfusion—refers to an intermittent flow disturbance, characterised by periods of normal flow interspersed with reduced flow, in the absence of any re-occlusion, White bars=Stable reperfusion—refers to the re-establishment of blood flow throughout a 60 min period, with no redevelopment of occlusion over this period.

FIG. 9. Effect of Rho Kinase Inhibitors in Combination with t-PA or Urokinase±Anticoagulants on Time Taken to Vascular Perfusion

Bar graphs (i) through (vi) demonstrate the time taken to establish reperfusion in a blocked blood vessel (where blood flow=0 mls/min), where reperfusion is described as the re-establishment of blood flow (where blood flow>0 mls/min). The various treatment regimens were as follows: A: saline, B: HA1077 (8 mg/kg), C: t-PA (2 mg/kg) bolus+18 mg/kg/30 min infusion, D: t-PA (2 mg/kg) & heparin (71 U/kg) boluses+t-PA (18 mg/kg/30 min) & heparin 28.6 U/kg/30 min infusion, E: t-PA (2 mg/kg) & heparin (142 U/kg) boluses+t-PA (18 mg/kg/30 min) heparin (57.2 U/kg/30 min) infusion, F: Y27632 (8 mg/kg) & t-PA (2 mg/kg) & heparin (142 U/kg) boluses+t-PA (18 mg/kg/30 min) & heparin (57.2 U/kg/30 min) infusion, G: HA-1077 (8 mg/kg) & urokinase (4,400 IU/kg) & heparin (142 U/kg) boluses+urokinase (4,400 IU/kg/30 min) & heparin (57.2 U/kg/30 min) infusion, H: HA1077 (8 mg/kg) & t-PA (2 mg/kg) & hirudin (10 mg/kg) boluses+t-PA 18 mg/kg/30 min) infusion.

### DETAILED DESCRIPTION OF THE INVENTION

Thrombosis describes the development of a blood clot (thrombus) in a blood vessel. Arterial thrombosis is a major clinical problem that most frequently manifests as a coronary thrombosis, leading to the occlusion of the coronary arteries and the development of an acute myocardial infarction (heart attack). Formation of thrombi within the deep veins of the lower extremities is characterised as deep vein thrombosis (DVT). Causative factors include immobilisation and venous stasis, hereditary and acquired prothrombotic states, oestrogen therapy and pregnancy. Certain surgical procedures also correlate strongly with postoperative venous clot formation. These include hip or knee replacement, elective neurosurgery, and acute spinal cord injury repair.

Therapeutic lysis of pathogenic thrombi is achieved by administering thrombolytic agents such as tissue plasminogen activator (t-PA). Benefits of thrombolytic therapy include rapid lysis of the thrombus with restoration of blood flow (reperfusion). Complications however include internal and external bleeding due to lysis of physiologic clots, leading to hemorrhagic stroke. Currently available thrombolytics, in addition to t-PA include reteplase, streptokinase, anistreplase, urokinase, and tenecteplase. Thrombolytic treatment of acute myocardial infarction is estimated to save 30 lives per 1000 patients treated; nevertheless, the 30-day mortality for this disorder remains substantial.

The efficacy of thrombolytic therapy in the treatment of myocardial infarction has been demonstrated over the past ten years using one or more of the agents described above. Unfortunately, there are side effects associated with these agents. For example, recombinant t-PA (marketed under various trade names ACTIVASE, CATHFLO ACTIVASE, ACTIVASE rt-PA, ACTILYSE) is associated with secondary toxicity such as hypofibrinogenemia and bleeding. Adverse reactions that have been associated with t-PA therapy include arrhythmia, heart failure, cardiac arrest, recurrent ischemia, myocardial reinfarction, pericarditis, thromboembolism, pulmonary edema, and hypotension. In addition, the rate at which fibrinolytics such as t-PA induce thrombolysis is highly variable, with ˜25% of patients harbouring thrombi resistant to lysis. Studies have reported the composition of the thrombus as a critical determinant in lysis sensitivity, with platelet-rich thrombi demonstrating a greater resistance to t-PA-mediated lysis¹³. As coronary thrombi are frequently platelet-rich, the role of platelets in inhibiting clot lysis may play an important role in the regulation of coronary thrombolysis. In support of this hypothesis, clinical trials have demonstrated enhanced incidence of reperfusion, reduction in mortality and secondary complications by combining thrombolytic and anti-platelet therapies^{14,15,16}.

A significant finding of the present invention is that the addition of a platelet contractility inhibitor to a thrombolytic and anticoagulant agent significantly enhanced the timing of reperfusion compared to the absence of the platelet contractility inhibitor. Time to reperfusion is a critical issue in the management of patients with acute thrombotic events, with reperfusion times of 30 minutes or more typically observed with thrombolytic therapy alone. Thrombotic reocclusion is also a limitation of thrombolytic therapy, resulting in reocclusion rates of approximately 25% in patients with acute myocardial infarction. The combination of a platelet contractility inhibitor with a thrombolytic agent±anticoagulant significantly reduces the rate of arterial occlusion following reperfusion. This clearly has benefit in the treatment and management of stroke and myocardial infarction.

The use of thrombolytic therapy for the treatment of pulmonary embolism is controversial. Despite the theoretic advantages of thrombolysis over standard therapy, little data supports its widespread use over standard anticoagulation therapy except in situations where it is truly indicated i.e. massive pulmonary embolism with hypotension or system hypoperfusion¹⁷. However, no evidence exists to show benefit of thrombolytic therapy over standard anticoagulation therapy for recurrent pulmonary embolism, mortality or chronic complications. Because most patients with hypotensive massive pulmonary embolism die within two hours of the onset of symptoms, the use of a Rho kinase inhibitor in this setting may allow for more effective thrombolysis with a lower dose thrombolytic and longer therapeutic treatment window.

By performing real-time analysis of platelet adhesion and aggregation on a collagen substrate, the present inventors have elucidated a distinct contractile phase to thrombus development (referred to as a “platelet thrombus contraction” phase) that occurs independently of thrombin generation and fibrin polymerisation.

The ability to reduce the contractile function of platelets may not only undermine the stability of forming thrombi, but may also increase the ability of thrombolytic agents, such as tissue-plasminogen activator (t-PA) or urokinase to lyse formed thrombi. Platelet contractility inhibitors such as inhibitors of Rho kinase or myosin II (Blebbistatin) have not previously been used to facilitate thrombus dissolution, as a role for Rho kinase and myosin II in promoting primary thrombus contraction has not been recognised.

In accordance with one embodiment of the invention described herein, the present inventors have found that the combination of a Rho kinase inhibitor together with either t-PA or urokinase thrombolytics agents and an anticoagulant agent work synergistically to facilitate thrombus lysis. In fact, the concentration of urokinase required at which synergy was achieved with the Rho kinase inhibitor and anticoagulant was found to be up to 100 times lower than that required to induce thrombolysis in a rodent pulmonary embolism model⁷. Accordingly, it is likely that doses of t-PA lower than the prescribed dose of 0.9 mg/kg can be used to treat acute ischemic stroke and hence reduce the frequency of side effects seen with administration of t-PA.

Currently, thrombolytic therapy can only be given to stroke patients within 3 hours of the onset of symptoms. If however, a platelet contractility inhibitor allows more effective thrombolysis with a lower dose of t-PA, the therapeutic time frame may be widened considerably.

### Thrombolytic Agents

### Tissue Plaminogen Activator

t-PA is currently the only approved drug for the management of acute ischemic stroke. The dosage of t-PA administered to an adult subject is dependent upon the condition being treated. The product information detailing the approved dosages and indications is publicly available from a pharmaceutical resource such as MIMS. For example, the recommended dosage for the treatment of acute ischemic stroke in an adult is intravenous (IV) administration at a dose of 0.9 mg/kg (max 90 mg) infused over 60 min with 10% of the total dose administered as an initial IV bolus over 1 min. For pulmonary embolism, the recommended dosage in adults is 100 mg intravenously administered over 2 hours, with heparin therapy initiated or reinstated near the end of or immediately following the t-TPA infusion when the partial thromboplastin time or thrombin time returns to twice normal or less. For acute myocardial infarction, the recommended dosage is based upon patient's weight and should not exceed 100 mg.

### Streptokinase

Streptokinase (streptase) has been indicated for the treatment of acute myocardial infarction, pulmonary embolism and deep vein thrombosis. The recommended dosage for acute MI in an adult is intravenous infusion of a total dose of 1,500,000 units within 60 min. For treatment of pulmonary embolism, DVT, arterial thrombosis or embolism, recommended treatment in adults is intravenous administration preferably within 7 days of a loading dose of 150,000 units infused into a peripheral vein over 30 minutes.

### Tenecteplase

Tenecteplase is indicated for the thrombolytic treatment of acute myocardial infarction. The recommended dosage is based on body weight and the administration is via IV bolus injection over 5-10 seconds. The maximum dose is 10,000 IU (50 mg). Tenecteplase has similar clinical efficacy to alteplase (rt-PA) for thrombolysis after myocardial infarction.

### Reteplase

Reteplase is indicated for thrombolytic therapy of acute myocardial infarction and is administered as a 10+10 U double bolus injection. 10 U of reteplase corresponds to 17.4 mg of reteplase protein mass.

### Anistreplase

Anistreplase is indicated for thrombolytic therapy of acute myocardial infarction. The recommended dosage is 30 units administered intravenously over two to five minutes.

### Urokinase

Urokinase has been indicated for the treatment of pulmonary embolism as well as clotted AV fistula and shunts and deep vein thrombosis. The recommended dosage for pulmonary embolism in an adult is a loading dose of 4,400 IU/kg over 10 minutes, followed by a maintenance dose of 4,400 IU/kg/hr over 12 hours.

Because the use of these thrombolytic agents is associated with a number of adverse events, but most particularly the risk of bleeding, and especially when administered with anticoagulants or agents that alter platelet function such as aspirin; methods which result in the use of significantly lower dosages of thrombolytics would be highly desirable.

### Platelet Contractility Inhibitors

### Rho Kinase Inhibitors

Rho kinase is a member of the myotonic dystrophy family of protein kinases and contains a serine/threonine kinase domain at the amino terminus, a coiled-coil domain in the central region and a Rho interaction domain at the carboxy terminus. Its kinase activity is enhanced upon binding to GTP-bound RhoA and when introduced into cells, it can reproduce many of the activities of activated RhoA. In smooth muscle cells Rho kinase mediates calcium sensitisation and smooth muscle contraction and inhibition of Rho kinase blocks 5-HT and phenylephrine agonist induced muscle contraction. When introduced into non-smooth muscle cells, Rho kinase induces stress fiber formation and is required for the cellular transformation mediated by RhoA. Rho kinase regulates a number of downstream proteins through phosphorylation, including myosin light chain, the myosin light chain phosphatase binding subunit and LIM-kinase 2.

Rho kinase inhibitors have found to be useful in the treatment of vascular disease including pulmonary hypertension, stable angina and atherosclerosis. In addition, Rho kinase inhibitors have been found to play a role in inhibiting tumor cell migration and anchorage-independent growth.

Various Rho kinase (ROCK) inhibitors have been described including Y-27632 ([(+)-(R)-trans-4-(1-aminoethyl)-N-(4-pyridyl)cyclohexanecarboxamide dihydrochloride] and Y-30141 ([(+)-(R)-trans-4-(1-aminoethyl)-N-(1H-pyrrolo[2, 3-1)]pyridine-4-yl)cyclohexanecarboxamide dihydrochloride] which are selective for p160ROCK (ROCK-I) and ROKα/Rho-kinase(ROCK-II) (Ishizaki T et al., 2000, Molecular Pharmacology 57:976-983).

Other Rho kinase inhibitors include H1152 (S)-(+)-2-methyl-1-[(4-methyl-5-isoquinolinyl)sulfonyl]homopiperazin, 2HCl also known as dimethylfasudil or HA-1077 1-(5-isoquinolinesulphonyl)-homopiperazine HCl also known as fasudil hydrochloride (Asano T et al., 1989, Br. J. Pharmacol. 98:1091-1100).

### FASUDIL®

Fasudil is currently the only approved Rho kinase inhibitor in clinical use. An intravenous formulation of Fasudil was approved in 1995 in Japan for the prevention of cerebral vasospasm in patients with subarachnoid hemorrhage.

Oral and inhaled formulations of Fasudil are being developed for the treatment of pulmonary arterial hypertension.

Various formulations of Fasudil have been described. For example, WO 2005/117896 describes a formulation of Fasudil in a matrix body and envelope comprising poly vinyl pyrrolidone and poly vinyl acetate. WO 2005/087237 describes an improved stabilised formulation of Fasudil and WO 2000/009133 describes an oral preparation of Fasudil hydrochloride. Such Fasudil containing formulations are considered to be suitable for use in the methods of the present invention.

In one embodiment, the Rho kinase inhibitor according to the invention is 1-(5-isoquinolinesulphonyl)-homopiperazine HCl (HA1077).

The term “derivatives having Rho kinase inhibitory activity” is intended to encompass active metabolites of Rho kinase inhibitors such as 1-(hydroxyl-5-isoquinoline sulfonyl-homopiperazine (hydroxyfasudil).

Additional Rho kinase inhibitors have also been described including isoquinolinesulfonamide derivatives such as those described in U.S. Pat. No. 4,634,770 and compounds described in U.S. Pat. No. 6,943,172, U.S. Pat. No. 6,924,290, U.S. Pat. No. 6,451,825, U.S. Pat. No. 6,906,061, U.S. Pat. No. 6,218,410.

Such derivatives are included within the scope of the claims of the present invention.

The Rho kinase inhibitor is administered in combination with one or more thrombolytic agents such as those described above. The Rho kinase inhibitor and thrombolytic agent may be administered sequentially or concurrently.

As the methods of the present invention are designed to facilitate thrombus dissolution at an early stage (within 12 hours of symptom onset from a thromboembolic event), the dose of thrombolytic agents that can be used in conjunction with Rho kinase are less than that typically used. For example, it is expected that the total dosage range for t-PA therapy of acute ischemic stroke in a human subject would be in the order of 5-90 mg.

### Blebbistatin

Blebbistatin (so named because of its ability to block cell blebbing) is a selective and high-affinity (IC₅₀ approx. 4 μM) inhibitor of non-muscle myosin II. During cell division blebbistatin inhibits contraction of the cleavage furrow without disrupting mitosis.

### Rho Family of GTPases

The Rho family of GTPases is a family of small signalling G proteins (GTPase) and is a subfamily of the Ras superfamily. The members of the Rho GTPase family have been shown to regulate many aspects of intracellular actin dynamics, and are found in all eukaryotic organisms including yeast and some plants. Rho proteins are involved in a wide variety of cellular functions such as cell polarity, vesicular trafficking, the cell cycle and transcriptomal dynamics.

### Anticoagulants

The methods of the present invention also encompass the use, where appropriate of one or more additional anticoagulant agents selected from the group consisting of warfarin, hirudin and heparin.

It will be appreciated by persons skilled in the art that additional anticoagulant agents not listed above will also be suitable for use in the present invention.

### Additional Agents

Additional agents may also be used in the methods of the present invention including, one or more agents selected from asprin, non-steroidal anti-inflammatory drugs (NSAIDs), abciximab, dipyridamole and, clopidogrel.

### Administration of Platelet Contractility Inhibitor

The platelet contractility inhibitor used in the methods of the invention is administered to the subject in an effective amount. Generally, an effective amount is an amount effective to cause dissolution of a forming thrombus without substantially increasing the risk of hemorrhage, as measured by a normal skin bleeding time.

The dosage administered depends upon the age, health and weight of the subject. Typically, the dose administered to the subject will be according to the prescribed information in the case of the Rho kinase inhibitor Fasudil.

Administration preferably occurs by bolus injection or by intravenous infusion, preferably as soon as possible after the identification of a thromboembolic event. For effective dissolution of a forming thrombus, the Rho kinase inhibitor should be administered approximately 0-12 hours after the first identification of a thromboembolic event.

The Rho kinase inhibitor can be administered by any suitable means, including, for example, parenteral or local administration, such as intravenous injection or direct injection into the thrombus, by oral administration or by inhalation. In a preferred form, the Rho kinase inhibitor is administered as an intravenous bolus injection or as an intravenous infusion. Bolus injection of the Rho kinase is preferably performed soon after thrombosis i.e. before admission to hospital.

The timing of administration of the platelet contractility inhibitor with the thrombolytic and optionally anticoagulant(s) will depend upon the thromboembolic event to be treated. For example for myocardial infarction, it would be preferred to administer the agents at the same time to the subject. For a stroke event, the preferred course would be to administer the platelet contractility inhibitor together with the thrombolytic agent. For local administration at the site of arterial thrombotic occlusion, concurrent administration of the platelet contractility inhibitor, thrombolytic and anticoagulant would be preferable.

The term “subject” is used herein is intended to refer to human subjects. However, the subject may also be a primate animal, or a domestic animal such as a dog, cat or horse.

### Example 1

### Materials and Methods

### Materials

The Rho kinase inhibitor 111152 was obtained from Toronto Research Chemicals (Canada). IP₃ receptor antagonist 2-aminoethoxydiphenyl borate (2-APB) was from Cayman Chemicals (Michigan, USA), HA1077, the myosin II inhibitor Blebbistatin [−] and it inactive enantiomer Blebbistatin [+] were obtained from Chemicon (USA). DiIC₁₂ was from BD Biosciences (NSW, Australia). Recombinant hirudin (Lepirudin) was purchased from Pharmion (Australia).

### Mouse Strains

All procedures involving the use of C57B16 and PAR4^{-/-} mice were approved by the Alfred Medical Research and Education Precinct (AMREP) animal ethics committee (AEC) (Melbourne, Australia), under project numbers E/0569/2007/M, E/0621/2007/M and E/0464/2006/M.

### Collection of Blood, Preparation of PRP and Washed Platelets

All procedures involving collection of human and mouse blood were approved by the Monash University Standing Committee on Ethics in Research involving Humans (SCERH) (Project number CF07/0125-2007/0005), and the Alfred Medical Research and Education Precinct (AMREP) animal ethics committee (AEC) (Melbourne, Australia) (SOP19—collection of whole blood from mice), respectively. For isolation of human platelet-rich plasma (PRP), whole blood from consenting healthy volunteers was collected into trisodium citrate (0.38% final concentration), and centrifuged at 300×g for 16 minutes at 37° C. Washed platelets were prepared from acid-citrate dextrose (ACD)-anticoagulated whole blood, with the inclusion of Lepirudin (800 U/ml) in the Platelet Washing Buffer and Apyrase (0.02 U/ml) in the Tyrode's Buffer.

### Visualisation and Quantification of In Vitro Thrombus Consolidation Under Flow.

Flow-based thrombus formation assays on a bovine fibrillar Type I collagen matrix were performed at 37° C. in the absence of fibrin. Briefly, anticoagulated (800 U/ml Lepirudin) human whole blood was preincubated with vehicle (DMSO), Blebbistatin (+) (200 μM), Blebbistatin (−) (200 μM), EGTA/Mg²⁺ (2 mM/1 mM), HA1077 (80 μM), H1152 (40 μM) or 2-APB (200 μM) (10 mins, 37° C.) prior to perfusion through fibrillar type I collagen-coated microcapillary tubes (2.0 mg/ml) at 1800 s⁻¹ for 5 mins. Thrombus formation was observed using an inverted Leica DMIRB microscope (Leica Microsystems, Wetzlar, Germany) with a 63× water objective (1.2 numeric aperture (NA)), and recorded in real-time using a Dage-MTI charge-coupled device (CCD) camera 300 ETRCX (Dage-MTI, Michigan City, Ind.).

Two-dimensional quantification of thrombus consolidation—Quantification of thrombus contraction was performed by ‘spiking’ whole blood with 3% DiIC₁₂-labelled platelets prior to perfusion. Spiked whole blood was then perfused over collagen matrices as described above, and DIC/fluorescence images were recorded in real-time as described above, for off-line analysis. Studies examining the effect of 2-APB on consolidation were performed by perfusing untreated whole blood across microslides for 30 seconds to establish a nucleating thrombus, followed by perfusion of inhibitor treated blood. This pre-inhibitor perfusion was necessary as the presence of 2-APB prevents thrombus formation, precluding the analysis of consolidation. The distance between 2 fluorescently-labelled platelets in a given thrombus was measured in mm every 30 sec over 5 min. Results are expressed as percentage decrease in the distance between 2 platelets incorporated into a thrombus prior to 1 minute of flow, with the distance between platelets at 1 minute taken as 100%.

3D volumetric thrombus analysis—For analysis of thrombus volume, whole blood was labelled with DiIC₁₂ (1 μM) prior to perfusion. Thrombi were formed as described above, and images captured in real-time using an inverted Leica DMIRB confocal microscope, with 1 μM sections acquired every 30 seconds over 4-5 min. Analysis of thrombus volume was performed using Metamorph 6 software.

### Intravital Microscopy

The development and consolidation of thrombi in response to vessel injury was monitored using intravital microscopy. C57BL6 or PAR4 deficient (15 g-18 g) mice were anaesthetised using sodium pentobarbitone (60 mg/kg), and the mesentery exteriorized through a midline abdominal incision. Body temperature was maintained during the procedure using an infrared heat lamp, and exposed mesenteric vessels (50-160 μm diameter) were hydrated using warm saline. Vessel injury was achieved either through vessel puncture using a microinjection needle (20-30 μm tip diameter) connected to a micromanipulator (Eppendorf), or through application of 6% FeCl₃-soaked filter paper (8 sec). Accrual of platelets to the area of injury was recorded in real-time as described for in vitro flow assays (above). In some experiments, H1152 (5 mM stock solution, 2.5 μl injection volume per cycle), HA1077 (10 mM stock solution, 2.5 μl injection volume per cycle), 2-APB (25 mM stock solution, 2.5 μl injection volume per cycle), Blebbistatin [−] or its inactive enantiomer Blebbistatin [+] (25 mM stock solution, 2.5 μl total injection volume), or an equivalent volume of vehicle (DMSO), were locally infused into developing thrombi via the microinjection needle (release rate 2-3 μl/min, 3 cycles). To prevent fibrin generation, in some experiments, lepirudin (50 mg/kg) was administered via intravenous injection prior to induction of injury and subsequent injection of inhibitors. Complete abolition of fibrin formation at this concentration of lepirudin was confirmed by histology using Carstair's stain. The surface area of thrombi in vivo was measured using Image J, with analysis of every 5^{th} frame (at 1 frame/sec) over 4-5 mins. Change in surface area was expressed as fold-increase or decrease over the original surface area (=1).

### Platelet-Mediated Fibrin Clot Retraction

Platelet-mediated fibrin-dependent clot retraction was measured using citrated PRP^{9,10}. Results are expressed as, the percentage of serum remaining in the tube following clot removal, after subtracting the volumes obtained for c7E3 (negative control) samples.

### Statistical Analysis

Statistical significance between multiple treatment groups was analysed using a 1-way ANOVA with Dunnett's multiple comparison test. Statistical significance between multiple treatment groups over time was performed using 2-way ANOVA, with bonferroni post-tests. Statistical significance between 2 treatment groups was analysed using an unpaired student t-test with 2-tailed p values (Prism software, GraphPAD Software for Science, San Diego, Calif.) (ns [not significant]; p>0.05; p<0.05; p<0.01; ***p<0.001). Data are presented as means±either SEM or SD (where indicated), where n=the number of independent experiments performed.

### Example 2

### Identification of a Contractile Phase During Thrombus Development

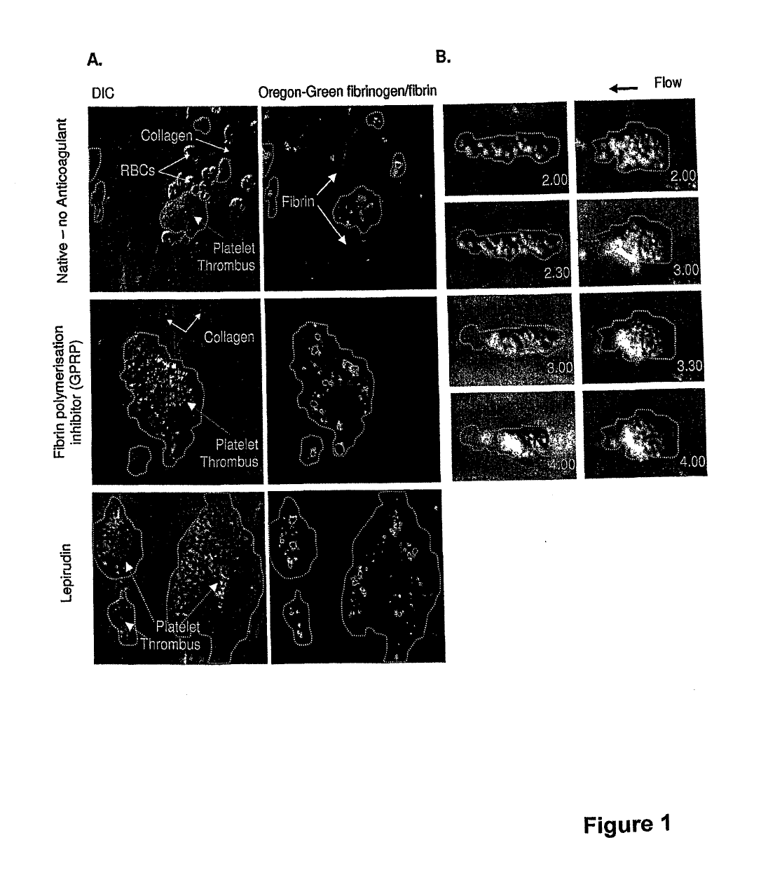

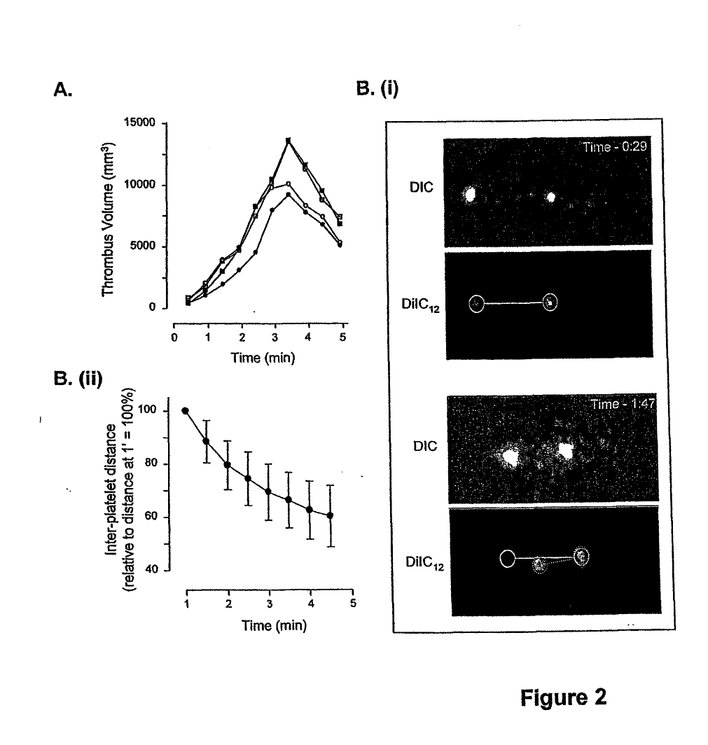

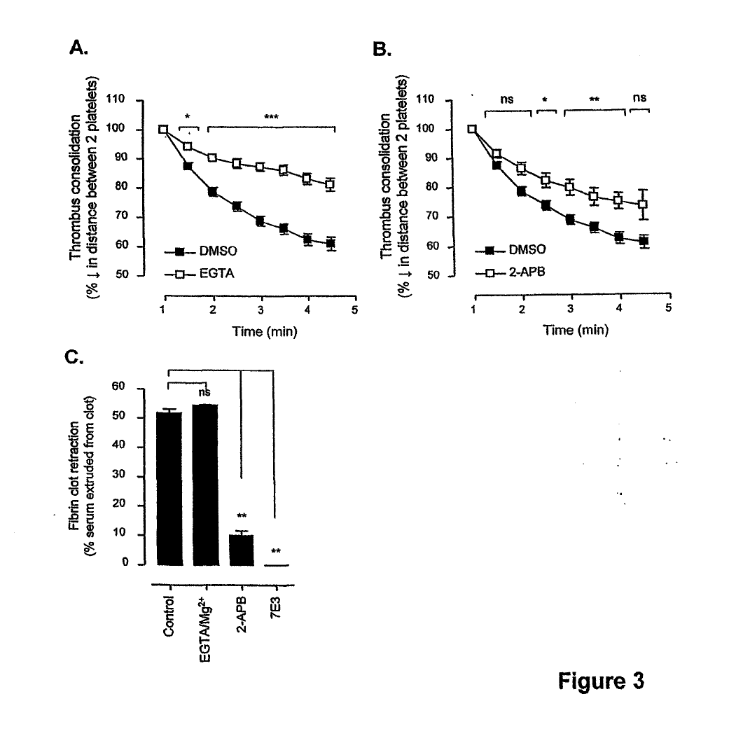

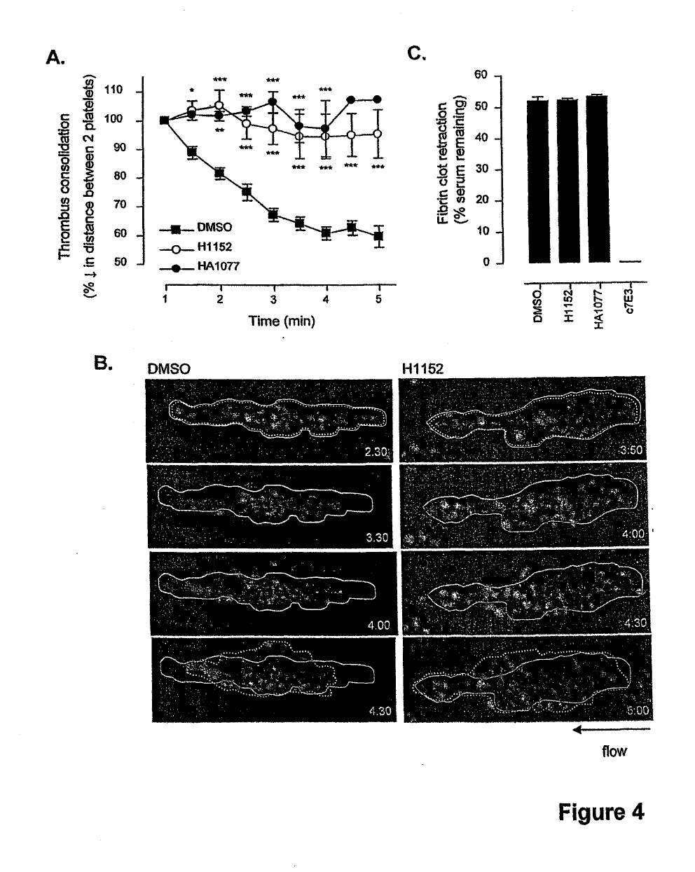

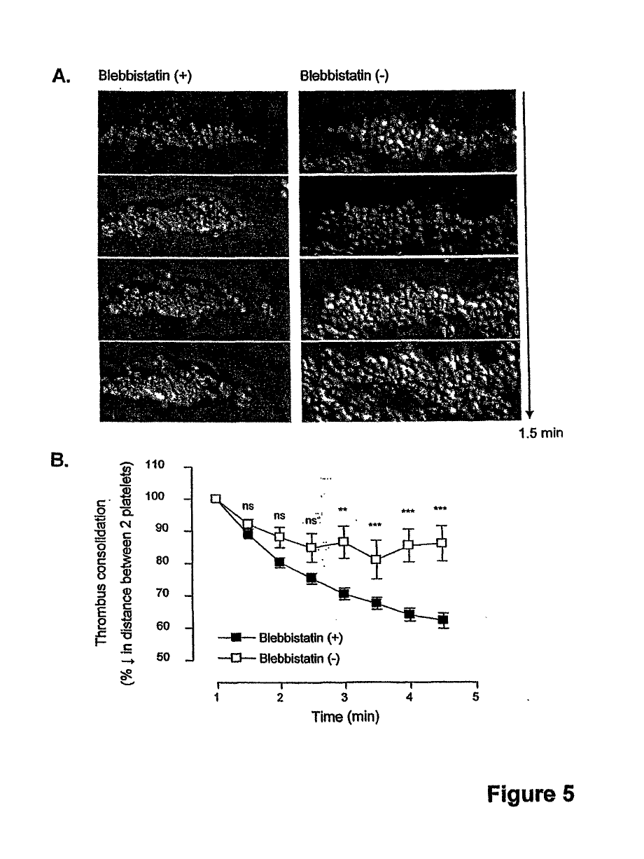

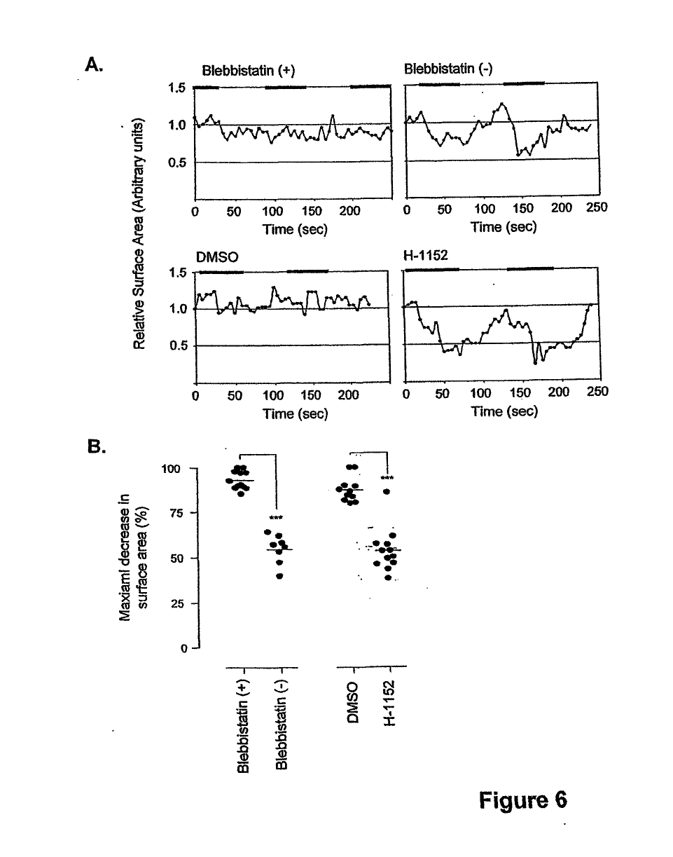

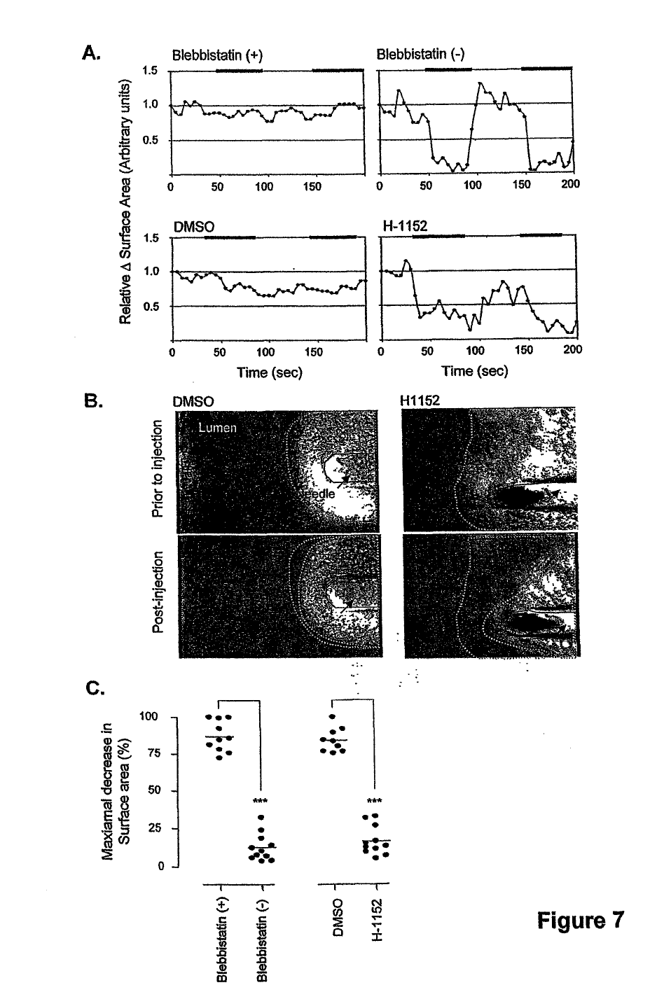

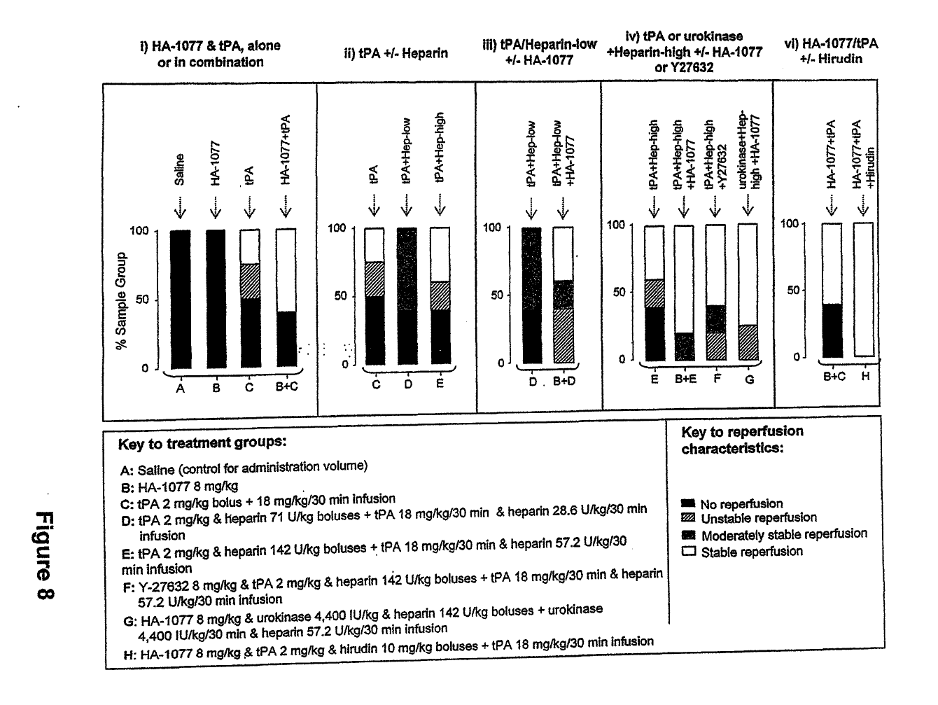

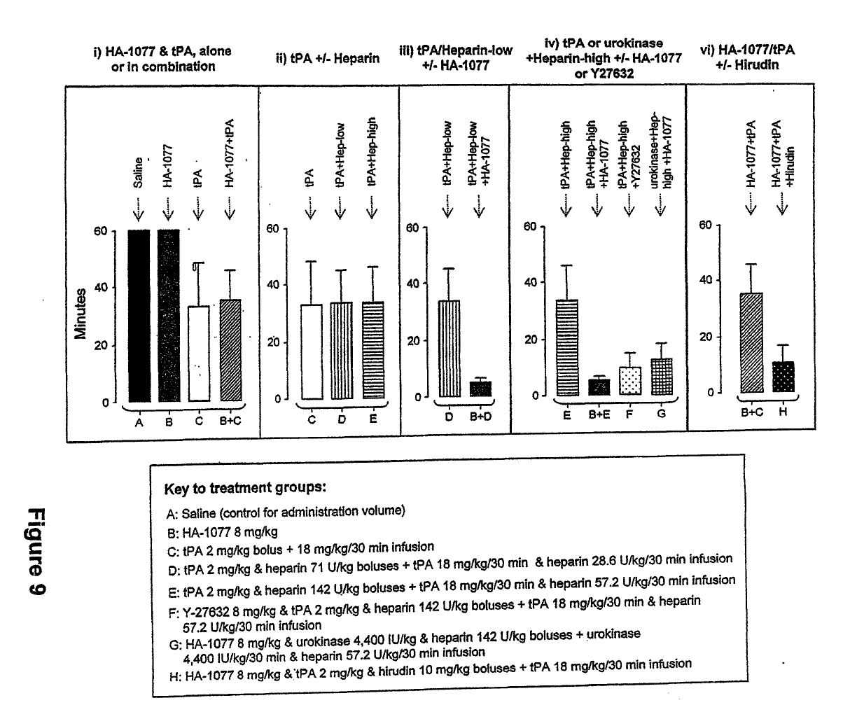

The importance of platelets in transmitting cytoskeletal contractile forces to fibrin polymers, leading to clot retraction, is well defined. However, the importance of these contractile mechanisms in regulating the various stages of thrombus growth, particularly under physiological blood flow conditions, has been less clearly defined. To investigate this, the inventors utilized an in vitro perfusion system that allows real-time analysis of platelet adhesion and thrombus growth on an immobilized Type I fibrillar collagen substrate. Perfusion of native (non-anticoagulated) whole blood at arteriolar shear rates (1800 s⁻¹) resulted in rapid platelet adhesion and aggregate formation, with the formation of large stable aggregates within 2 minutes of flow. Analysis of deposited fibrin(ogen), by co-perfusing fluorescently labelled fibrinogen, revealed widespread fibrin(ogen) incorporation within the developing thrombus, with individual thick fibrin strands prominent around the base of thrombi and over the collagen surface (FIG. 1A). Concomitant with thrombus formation, a time-dependent contraction of thrombi was observed. Contraction of thrombi was apparent within the first 60 seconds of flow and was continuous throughout the 5 min perfusion period. High resolution imaging of thrombi revealed that thrombus contraction was associated with the progressive tight packing of aggregated platelets, such that the margins of individual platelets could no longer be distinguished within the developing thrombus. Notably, retraction of platelets into the developing thrombus occurred prior to the development of thick fibrin polymers, raising the possibility that this process occurred independent of fibrin polymerization. To investigate this, the inventors performed perfusion studies in the presence of the Gly-Pro-Arg-Pro peptide (GPRP), an inhibitor of fibrin polymerization. The addition of GPRP to native whole blood inhibited the formation of individual fibrin polymers but had no inhibitory effect on platelet thrombus growth (FIG. 1A). Furthermore, the contraction of platelet thrombi was unaltered by GPRP. Similarly, thrombi formed using hirudin-anticoagulated whole blood also underwent a prominent contractile phase, leading to marked consolidation of forming thrombi (FIG. 1B). To exclude the possibility that trace amounts of thrombin were responsible for this contractile process, the inventors performed studies on mouse platelets that are completely unresponsive to thrombin stimulation (PAR4^{-/-} mice). PAR4 deficiency had no inhibitory effect on thrombus contraction or on the consolidation of forming thrombi. Moreover, treating whole blood with very high concentrations of lepirudin (1600 U/ml), in combination with the low molecular weight heparin, enoxaparine (400 U/ml), also did not prevent thrombus contraction, confirming that this phenomenon occurred independent of thrombin generation and fibrin polymerization.

To determine the impact of contraction on the volume of forming thrombi, 3-D volumetric analysis of thrombi was performed. Hirudin-treated whole blood preincubated with the fluorescent membrane dye DiIC₁₂ was perfused over Type I collagen at 1800 and confocal sections of developing thrombi taken at 30 second intervals over a 5 minute time period. Thrombi were reconstructed in 3-dimensions and volume quantified as described under Materials and Methods. As demonstrated in FIG. 2A, the volume of individual thrombi increased in a time-dependent manner (volume of individual thrombi ranging from ˜5,000 mm³ up to 15,000 mm³), with maximal thrombus size apparent after 3-3.5 minutes of flow. Contraction occurred continuously throughout thrombus development, however it was not until after 3.5 minutes of flow that a net decrease in thrombus volume was apparent, with an overall decrease between 23.9-48.2% (mean 38.2+/−16.1% S.D. n=10). Thrombus contraction typically involved the retraction of individual platelets into the body of the developing thrombus with the most rapid contraction occurring in the downstream tail of the developing thrombus (FIG. 1B). To quantify the change in distance between individual platelets during thrombus contraction, the inventors established a fluorescence-based tracking method that enabled analysis of movement of individual platelets following stable incorporation into thrombi (FIG. 2Bi, see under ‘Materials and Methods’). These studies revealed a time-dependent reduction in the distance between individual platelets, ranging from 12.5-62.5% (mean 37.7+/−12.8% S.D. n=36) (FIG. 2B ii).

### Example 3

### Importance of Rho Kinase for Thrombus Contraction

Actinomyosin-based contractility is tightly linked to the phosphorylation of myosin light chain kinase, through calcium/calmodulin-dependent activation of myosin light chain kinase and Rho kinase-dependent inactivation of myosin phosphatise. In platelets, calcium-activation of myosin light chain kinase appears to be the dominant contractile mechanism regulating platelet shape change and fibrin clot retraction⁴. To investigate the role of cytosolic calcium flux in regulating thrombus contraction, whole blood perfusion studies were performed under experimental conditions preventing calcium influx (EGTA/MgCl₂) or calcium mobilization from internal stores (IP₃ receptor antagonist—2-APB). Chelating extracellular calcium with EGTA significantly reduced the rate of thrombus contraction (up to 52% at 5 mins perfusion p<0.001, FIG. 3A). Under similar assay conditions, inhibition of calcium mobilization (2-APB) had a less pronounced effect on thrombus contraction, reducing contraction by 32% (FIG. 3B). This contrasted markedly with fibrin clot retraction, in which EGTA/MgCl₂ had no significant inhibitory effect (FIG. 3C) whereas APB abolished clot retraction at all time points examined (FIG. 3C).

To examine the contribution of Rho kinase to thrombus contraction, the effects of the Rho kinase inhibitor H1152¹¹ were examined. H1152 had a marked effect on the thrombus contraction process, resulting in an 88% decrease after 5 mins perfusion (p<0.001, FIG. 4A, B). This defect in contraction was associated with reduced tight packing of platelets into the developing thrombus, leading to the formation of less stable thrombi (FIG. 4B). Similar findings were obtained with another Rho kinase inhibitor HA1077 (FIG. 4A). These effects were selective to thrombus contraction, as neither inhibitor had any significant effect on the rate and extent of fibrin clot retraction (FIG. 4C).

### Example 4

### Inhibiting Platelet Contractility Undermines the Stability of Platelet Thrombi

The ability of the platelet contractile apparatus to promote tight packing of platelets within a developing thrombus suggests a potentially important role for contractility in maintaining thrombus stability. To investigate the importance of platelet contractility in this process we examined the effect of the myosin IIa inhibitor, blebbistatin on thrombus growth and stability. As demonstrated in FIG. 5, blebbistatin-treated platelets were able to adhere and form large aggregates on the Type I fibrillar collagen substrate, however the subsequent tight packing of platelets did not occur, leading to the development of highly unstable platelet thrombi. This lack of thrombus consolidation resulted in continual embolization of platelets from the thrombus surface, undermining the growth of forming thrombi (FIG. 5). To determine whether platelet contractility is important to maintain thrombus stability in vivo, the inventors established an intravital thrombosis model in the mouse microcirculation that enables real-time dynamic analysis of thrombus growth and stability. In this model, platelet thrombi are induced in post-capillary venules by micropuncture of the vessel wall with a microinjector needle. Non-occlusive thrombi rapidly form at the site of injury and high magnification imaging revealed that thrombi formed under these conditions primarily consisted of platelets. Consistent with this, pretreating mice with a platelet GPIb receptor antagonist (alboaggregin) or GPIIb-IIIc antagonist (GPI-162) completely eliminated thrombus formation. High magnification imaging of forming thrombi revealed the progressive tight packing of individual platelets within the core of the developing thrombus that was associated with thrombus contraction. Similar findings were apparent with thrombi formed following FeCl₃ induced vascular injury, suggesting that thrombus contraction represented a general feature of thrombus growth in vivo. The local administration of blebbistatin into the microcirculation following thrombus formation resulted in the loss of tight packing between individual platelets, particularly in the outer layers of formed thrombi, leading to progressive embolization of platelet aggregates from the thrombus surface (FIG. 6A) and a mean reduction in thrombus size by 38% (FIG. 6B). In control studies, microinjection of vehicle alone or the inactive blebbistatin enantiomer had no effect (FIGS. 6A and B). Furthermore, with cessation of blebbistatin administration, thrombi rapidly reformed at the site of injury such that repetitive cycles of thrombus growth and embolization could be achieved with regular cycles of blebbistatin administration (FIG. 6A).

To investigate the role of Rho kinase in regulating thrombus stability, H1152 was locally administered into the microcirculation following thrombus development. Identical to the findings with blebbistatin, inhibiting Rho kinase undermined the sustained tight packing of aggregated platelets, particularly in the superficial layers of thrombi, leading to embolization of platelets from the thrombus surface (FIG. 6A) and a mean reduction in thrombus size by 34% (FIG. 6B). In control studies, the local administration of vehicle (DMSO) control was without effect (FIGS. 6A and B). Rho kinase appeared to play the dominant role in this process, as H1152 was more effective than the IP₃ receptor antagonist APB at inducing thrombus instability and embolization. These studies define a major role for Rho kinase and the platelet contractile mechanism in maintaining thrombus stability in vivo.

### Example 5

### Platelet Contractility is Essential for the Stability of the Primary Hemostatic Plug

To investigate whether thrombus contraction in vivo required thrombin stimulation of platelets, intravital microscopy studies were performed on PAR4^{-/-} mice. The initial platelet adhesion and aggregation response of these platelets was normal following micropuncture of post-capillary venules, however the thrombi that formed were less stable than PAR4^{+/+} controls, leading to repetitive cycles of thrombus formation and embolization, particularly in the superficial layers of forming thrombi. These findings confirm previous reports that thrombin-stimulation of platelets plays a critical role in stabilizing forming thrombi¹². Despite their instability, thrombi formed in PAR4^{-/-} mice underwent a prominent contractile phase that led to thrombus consolidation, particularly in the core of forming thrombi.

To eliminate thrombin, and thereby exclude the possible involvement of fibrin clot retraction to this process, wild type mice were pretreated with high dose lepirudin (50 mg/kg) prior to vessel injury. Mural thrombus formation occurred rapidly following needle puncture of post-capillary venules, however in the absence thrombin, thrombi were more unstable, leading to continuous embolization of platelet aggregates from the thrombus surface. Nonetheless, despite persistent surface embolization, a stable thrombus core eventually developed (typically within 3-4 min post-injury) that was of sufficient stability to resist the detaching effects of rapid blood flow over a 15 minute observation period. Local infusion of the active enantiomer of blebbistatin resulted in rapid destabilization of the primary hemostatic plug, with near complete embolization of the formed thrombus (FIG. 7A). Similarly, inhibition of Rho kinase produced a similar defect in the stability of the primary hemostatic plug, with embolization occurring within 10-15 seconds of drug infusion (FIG. 7A-C). In control studies, injection of the vehicle (DMSO) or the inactive blebbistatin enantiomer had no adverse effect on the stability of the primary hemostatic plug (FIG. 7A, C). Taken together, these findings suggest a major role for Rho kinase-dependent platelet contractility in maintaining the integrity of the primary hemostatic plug, independent of thrombin and fibrin polymers.

### Example 6

### Effect of Rho Kinase Inhibitors in Combination with t-PA (or Urokinase) and with or without Anticoagulants on Vascular Perfusion

Mice were anaesthetised and minor surgery performed to expose to the carotid artery and jugular vein. A Doppler flow probe was placed around the carotid artery to monitor blood flow through this blood vessel, and a catheter placed in the jugular vein to administer drugs. A blood clot was formed in the carotid artery of the mouse through the delivery of a small electric current (4 mA for 1.25 min^{g}), resulting in complete blockage of blood flow through the vessel (blood flow=0 mls/min), as measured by the flow probe. After vessel blockade was established, various combinations of t-PA, urokinase, heparin, hirudin, HA1077 and Y27632 were administered, using concentrations and regimens indicated (A-H in FIGS. 8 and 9) and examined for their efficacy to dissolve blood clots and restore blood flow. Blood flow was monitored in mice receiving each drug combination for a further 60 minutes, and blood flow measurements recorded using computer software.

The data presented in FIGS. 8 and 9 demonstrate that the thrombolytic agent t-PA was relatively moderate in its ability to lyse occlusive blood clots (refer treatment group C). This suggests that t-PA is only able to effect a partial lysis of blood clots and do not effectively prevent re-occlusion of the clots. The Rho kinase inhibitor HA1077 when used alone were unable to lyse blood clots and re-establish blood flow (refer treatment group B). It was also found that the combination of anticoagulant agent heparin with t-PA caused a dose dependent increase in clot lysis, consistent with the ability of thrombin inhibitors to prevent reformation of the fibrin blood clot.

Of significance, it was found that the combination of Rho kinase inhibitor with t-PA enhanced clot lysis in a synergistic manner (refer treatment group B+C).

In addition, the combined administration of a Rho kinase inhibitor, together with t-PA or urokinase and heparin or hirudin, was found to further enhance clot lysis in a synergistic manner, over and above that observed for the combination of Rho kinase inhibitor and thrombolytic agent. Using this combination therapy, blood flow was restored in all animals tested (refer treatment groups B+D, B+E, F, G, and H).

Furthermore, also of significance was the finding that the combined administration of a Rho kinase inhibitor, together with t-PA or urokinase and heparin/hirudin significantly decreased the time taken to establish reperfusion (refer treatment groups B+D, B+E, F, G and H), when compared with t-PA alone or t-PA and heparin.

Accordingly, the addition of a Rho kinase inhibitor to standard clot busting therapies enhances the efficacy of these drugs in a synergistic manner.

### CONCLUSIONS

These studies show that extracellular transmission of contractile forces plays an important role in promoting thrombus contraction, independent of thrombin and fibrin formation. In contrast to fibrin clot retraction, platelet thrombus contraction is principally regulated by Rho kinase-dependent signalling mechanisms. Furthermore, it is demonstrated that inhibition of thrombus contraction with blebbistatin or Rho kinase antagonists markedly undermines the stability of forming thrombi, leading to rapid embolization of the primary hemostatic plug. These studies suggest that during hemostasis platelets utilize a two-stage contractile mechanism: (i) initially involving the Rho kinase-dependent contraction and maintenance of the primary hemostatic plug; (ii) followed by fibrin generation and the calcium-dependent retraction of the secondary hemostatic plug.

Rho kinase-dependent contractility appears critical for the bundling of actin filaments, a process that applies tension to integrin bonds, inducing receptor clustering and recruitment of integrins into focal adhesion sites. A small number of Rho-dependent focal adhesion-like complexes develop in spread platelets however these structures do not appear to be essential for the transmission of contractile forces to fibrin polymers. It is possible that Rho-dependent clustering of integrin bonds plays an important role in strengthening cell-cell adhesion contacts, necessary for the development of stable platelet aggregates that can resist the detaching effects of high shear. Such high avidity adhesive interactions appear to be less critical for clot retraction, particularly when studied under non-sheared conditions, providing a potential explanation for the lack of involvement of Rho kinase in this process.

A striking effect in the in vivo models was the rapidity in which platelet thrombi embolize following exposure to inhibitors of platelet contractility, particularly under experimental conditions limiting thrombin generation and fibrin formation. The platelet adhesion contacts within the primary hemostatic plug are intrinsically unstable, requiring fibrin generation to stabilize the formed aggregates to secure hemostasis. The in vivo results presented here indicate that in the absence of contractility, primary hemostatic plugs are extremely unstable, becoming detached from the site of injury within seconds of exposure to contractility inhibitors. This suggests that there may be two distinct phases to stabilizing the primary hemostatic plug; the first is a rapid phase linked to platelet contractility and the physical tightening of platelet-platelet adhesion contacts; and the second; a slower phase linked to thrombin generation and fibrin polymerization. Such a two-stage stabilization process provides a dynamic mechanism of temporal control of thrombus growth and stability.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

1. Daniel J L, Molish I R, Rigmaiden M, Stewart G. Evidence for a role of myosin phosphorylation in the initiation of the platelet shape change response. J Biol. Chem. 1984; 259:9826-9831.
2. Flaumenhaft R. Molecular basis of platelet granule secretion. Arterioscler Thromb Vasc Biol. 2003; 23:1152-1160.
3. Cohen I. The contractile system of blood platelets and its function. Methods Achiev Exp Pathol. 1979; 9:40-86.
4. Leng L, Kashiwagi H, Ren X D, Shattil S J. RhoA and the function of platelet integrin alphaIIbbeta3. Blood. 1998; 91:4206-4215.
5. Schoenwaelder S M, Hughan S C, Boniface K, et al. RhoA sustains integrin alpha IIbbeta 3 adhesion contacts under high shear. J Biol. Chem. 2002; 277:14738-14746.
6. Calaminus S D, Auger J M, McCarty O J, Wakelam M J, Machesky L M, Watson S P. MyosinIIa contractility is required for maintenance of platelet structure during spreading on collagen and contributes to thrombus stability. J Thromb Haemost. 2007; 5:2136-2145.
7. Clozel J P, Holvet P, Tschopp T. Experimental pulmonary embolus in the rat: a new in vivo model to test thrombolytic drugs. J Cardiovasc Pharmacol. 12(5):520-525, 1988
8. Jackson S P, Schoenwaelder S M, Goncalves I, Nesbitt W S, Yap C L, Wright C E, Kenche V, Anderson K E, Dopheide S M, Yuan Y, Sturgeon S A, Prabaharan H, Thompson P E, Smith G D, Shepherd P R, Daniele N, Kulkarni S, Abbott B, Saylik D, Jones C, Lu L, Giuliano S, Hughan S C, Angus J A, Robertson A D, Salem H H. PI 3-kinase beta: a new target for antithrombotic therapy. Nature Medicine, 11(5):507-514.
9. Schoenwaelder S M, Jackson S P, Yuan Y, Teasdale M S, Salem H H, Mitchell C A. Tyrosine kinases regulate the cytoskeletal attachment of integrin alpha IIb beta 3 (platelet glycoprotein IIb/IIIa) and the cellular retraction of fibrin polymers. J Biol. Chem. 1994; 269:32479-32487.
10. Schoenwaelder S M, Yuan Y, Cooray P, Salem H H, Jackson S P. Calpain cleavage of focal adhesion proteins regulates the cytoskeletal attachment of integrin alphaIIbbeta3 (platelet glycoprotein IIb/IIIa) and the cellular retraction of fibrin clots. J Biol. Chem. 1997; 272:1694-1702.
11. Pandey D, Goyal P, Siess W. Lysophosphatidic acid stimulation of platelets rapidly induces Ca2+-dependent dephosphorylation of cofilin that is independent of dense granule secretion and aggregation. Blood Cells Mol. Dis. 2007; 38:269-279.
12. van Gestel M A, Reitsma S, Slaaf D W, et al. Both ADP and thrombin regulate arteriolar thrombus stabilization and embolization, but are not involved in initial hemostasis as induced by micropuncture. Microcirculation. 2007; 14:193-205.
13. Jang, I K et al., (1989) Circulation, 79:920-928.
14. Strategies for Patient Enhancement in the Emergency Department (SPEED) Group, Circulation, (2000) 101:2788-2794,
15. ISIS-2 (second international study of infract survival) collaborative group, The Lancet, (1988), 13:349-360;
16. The GUSTO V Investigators, The Lancet, (2001); 357-1905-1914.
17. Almoosa K. (2002) American Family Physician 65(6):1097-1102.

1-21. (canceled)

22. A method of treating a thromboembolic disorder, comprising administering to a subject a platelet contractility inhibitor, in combination with one or more thrombolytic agents and optionally one or more anticoagulants.

23. A method according to claim 22, wherein treating the thromboembolic disorder comprises dissolving a thrombus in a subject.

24. A method according to claim 22, wherein treating the thromboembolic disorder comprises inhibiting thrombus contraction in a subject.

25. A method according to claim 22, wherein the thromboembolic disorder is selected from the group consisting of, but not limited to, ischemic stroke, acute myocardial infarction, deep vein thrombosis (DVT), pulmonary embolus, clotted AV fistula and shunts.

26. A method according to claim 22, wherein the throboembolic disorder is a stroke or heart attack.

27. A method according to claim 22, wherein the platelet contractility inhibitor is administered locally at the site where the thrombus has formed.

28. A method according to claim 22, wherein the platelet contractility inhibitor is administered directly into the thrombus.

29. A method according to claim 22, wherein the platelet contractility inhibitor is administered to the subject within 12 hours after the first identification of a thromboembolic disorder.

30. A method according to claim 22, wherein the platelet contractility inhibitor is administered to the subject within 3 hours after the first identification of a stroke.

31. A method according to claim 22, wherein the platelet contractility inhibitor is administered sequentially or concurrently with the one or more thrombolytic agents and optionally one or more anticoagulants.

32. A method according to claim 22, wherein the platelet contractility inhibitor is selected from the group consisting of a Rho kinase inhibitor, blebbistatin and a Rho inhibitor.

33. A method according to claim 32, wherein the Rho kinase inhibitor is selected from the group consisting of:
(i) Isoquinolinesulfonamides such as (S)-(+)-2-methyl-1-[(4-methyl-5-isoquinolinyl)sulfonyl]homopiperazine (dimethylfasudil) or 1-(5-isoquinolinesulfonyl)homopiperazine (fasudil) or salts thereof;
(ii) (+)-(R)-trans-4-(1-aminoethyl)-N-(4-pyridyl)cyclohexanecarboxamide or salts thereof;
(iii) (+)-(R)-trans-4-(1-aminoethyl)-N-(1H-pyrrolo[2,3-b]pyridine-4-yl)cyclohexanecarboxamide] or salts thereof; or
(iv) derivatives having Rho kinase inhibitory activity.

34. A method according to claim 33 wherein the Rho kinase inhibitor is 1-(5-isoquinolinesulfonyl)homopiperazine hydrochloride (fasudil hydrochloride).

35. A method for enhancing the effectiveness of a thrombolytic agent, comprising administering to the subject a platelet contractility inhibitor together with the thrombolytic agent at a time when the thrombus is forming or has formed from aggregated platelets.

36. A composition for use in dissolving a thrombus, the composition comprising a platelet contractility inhibitor and one or more thrombolytic agents and optionally one or more anticoagulants.
